(19) United States
(12) Patent Application Publication
(10) Pub. No.: US 2007/0117085 A1
(43) Pub. Date: May 24, 2007

(54) Method and device for culturing live cells by coupling a bioreactor receiver with a selection automation

(75) Inventors: Jean Barthomeuf, Vincennes (FR); Veronique Jactat, Rennes (FR); Yann Beaujouan, Nanterre (FR)

Correspondence Address: BROWDY AND NEIMARK, P.L.L.C.;624 NINTH STREET, NW, SUITE 300, WASHINGTON, DC, 20001-5303, US

(73) Assignee: ECO SOLUTION, Romainville (FR)

(21) Appl. No.: 10/574,197

(22) PCT Filed: Sep. 30, 2004
(86) PCT No.: PCT/FR04/02476

§ 371 Date: Mar. 31, 2006

(30) Foreign Application Priority Data
Oct. 1, 2003 (FR) 0311502

(51) Int. Cl. C12Q 3/00; C12M 1/14; C12M 1/36

(52) U.S. Cl. 435/3; 435/286.1; 435/299.1

(57) ABSTRACT

The invention relates to a method for continuously, semi-continuously or discontinuously treating a substrate (24) consisting in placing said substrate in a bioreactor receiver (1) and in exposing it to the action of a living cell culture (C1) which makes it possible to carry out a reaction (R1) on the substrate (24) and to which a medium is periodically inoculated with the aid of living cells (C2) improving said reaction. Said living cells (C2) are obtainable by selection from a population of dynamic living cells carried out by an automatic selection device (2) which is supplied by the same substrate (24) as the bioreactor receiver (1) and is originally inoculated with the living cells (C1) contained in the bioreactor receiver (1) tank and an operating device.

The present invention relates to a method and a device for culturing living cells by coupling a bioreactor vessel to an automatic selection device.

Waste treatment is becoming an increasing constant preoccupation of the public and governments alike. A significant proportion of the treatments is carried out in factories utilizing tanks inoculated with a bacterial flora. But the bacterial flora changes over time and this change is often unfavourable to the reactions which are desired to occur in the tanks.

The problem of the drift of bacterial cultures is a general problem found for example in the pharmaceutical industry. In this industry, drift is avoided by operating in a sterile manner.

But it is economically inconceivable to work under such conditions for example in a waste-treatment factory.

The culture devices as utilized in the industry for the production of metabolites of commercial interest, or the biodegradation of waste or sewage for example, are faced with the problem of contamination by species originating from the external medium. The management of cultures under sterile conditions which involve the total confinement of the equipment is one solution to the problem of contamination by external species, but it is difficult to envisage for reasons of cost of treatment in applications such as the biodegradation of waste, or even impossible to implement in extensive utilizations of microbial populations such as in lagooning for example.

Moreover, WO 00/34433 describes a technique which allows the selection and accelerated proliferation of living cells in suspension. By maintaining constant cell concentration conditions (turbidostat) over unlimited periods, the device described therefore acts as an automated selection method which at the same time eliminates the static variants of living cells, i.e. the living cells which stagnate in the pipes and vessels and favours the dynamic variants remaining in suspension, which are increasingly well-adapted to the culture conditions.

The industrialization of such a device with a view to treating volumes of several m³, or even several tens or hundreds of m³ can be envisaged by simply scaling up, but its operation would thereby be made expensive for several reasons:
the means utilized for the periodic transfer from one container to the other as well as the periodic transfer of the sterilizing and rinsing liquids and culture additives consume energy,
a significant consumption of sterilizing and rinsing liquids,
the use of one tank at a time, the other remaining on standby therefore not being used in the method. It must however possess all the equipment necessary for the development of the culture: temperature regulation device, sterilizing system, stirring system, etc.
the difficulty of continuously reading the turbidity in a large bioreactor vessel with significant culture densities.

For these reasons, the biodegradation of sewage currently utilizes various bacterial populations the nature of which escapes the operator's control, and without it being possible to use only the species selected for their performance or their effectiveness with respect to the substrate, i.e. to the compounds to be degraded present in the sewage.

It would therefore be desirable to have a technique in particular for biodegradation of sewage making it possible to utilize essentially the species selected for their performance or their effectiveness relative to the compounds to be degraded present in the sewage.

Following prolonged research the Applicant has discovered a method making it possible to reproduce and control the proliferation conditions in a large bioreactor vessel, or in a natural medium such as a lagoon or a reservoir for example, and operating in a continuous, semi-continuous or discontinuous manner, without having recourse to confinement and sterilization, and favouring the dynamic variants of living cells which are increasingly well-adapted to the culture conditions.

This method is essentially based on the cooperation between a bioreactor vessel and an automatic device for selecting living cells.

This is why a subject of the present Application is a method for the continuous, semi-continuous or discontinuous treatment of a substrate, in which said substrate installed in a bioreactor vessel is subjected to the action of a culture of living cells C1 making it possible to carry out a reaction R1 on said substrate and in which the medium is inoculated periodically and preferably regularly using living cells C2 improving said reaction, said living cells C2 originating from a selection carried out by an automatic selection device, preferably exclusively in suspension, of a population of dynamic living cells and said automatic device for selecting living cells being supplied either by a different substrate or by the same substrate as the bioreactor vessel and being inoculated at the outset by the living cells C1 present in the tank of the bioreactor vessel, and in which advantageously living cells are removed from the tank of the bioreactor vessel in order to be transferred into the automatic selection device.

If desired, other living cells can at any time be added to the selection device or to the bioreactor vessel, for example in order to increase the cell concentration or to introduce new species.

In the present Application and in what follows, the term “dynamic living cells” denotes living cells proliferating in suspension and subjected to a directed selection (in contrast to “static living cells”, denoting living cells adhering to the surface of the vessels and pipes, thus escaping selection). The static living cells are advantageously periodically eliminated.

Generally, living cells proliferating in suspension and subjected to a directed selection will be used as living cells C2. In certain applications, not dynamic living cells, but static living cells will be used as living cells C2.

The substrate allows maintenance of the cultures of living cells C1, C2, etc.

Under preferential conditions for implementing the invention, the automatic device for selecting the dynamic living cells comprises:
two or more vessels making it possible to receive and maintain cultures of living cells in suspension,
a set of means making it possible to separately supply these vessels with sterilizing, cleaning or neutralizing liquids,
a set of means making it possible to supply these vessels with gas,
a set of means making it possible to supply these vessels with substrate,
a set of means making it possible to transfer the content of one vessel into the other and vice-versa,
a set of means making it possible to evacuate all or part of the content of these vessels to another device such as a bioreactor vessel,
a set of means making it possible to evacuate all or part of the content of these vessels to a refuse bin.

At the start of the implementation, living cells C1 are present in the bioreactor vessel tank and in the automatic selection device. Over time, the selection device favours (selects) the occurence and proliferation of variants of dynamic living cells C2, which are still better adapted to the culture conditions and counter-selects the less adapted living cells C1. The living cells C2 are transferred to the bioreactor vessel where they come into competition with the living cells C1 then supplant them. Finally, it is noted that the population of living cells C1 has been replaced by the living cells C2. Preferably, in parallel, living cells are collected from the bioreactor vessel in order to be transferred to the automatic selection device.

The automatic device for selecting dynamic living cells comprises in particular
(a) at least one first and at least one second culture vessel intended to receive a culture;
(b) a source of gas;
(c) a source of medium;
(d) a source for a sterilizing agent; and
(e) a system of pipes comprising means for connecting either one of the two culture vessels to the source of medium such as valves as well as for connecting the two culture vessels to each other and for connecting either of the other culture vessels to the source of the sterilizing agent.

The gas used can be adapted to aerobic or anaerobic living cells.

Under other preferential conditions of implementation of the invention, two connecting pipes are provided between the two culture vessels which comprise a common pipe section.

Under other preferential conditions of implementation of the invention, there is provided on the common pipe section an evacuation pipe through which cultures can be collected from the culture vessels. The living cells C2 improving the reaction, are preferably collected via this pipe.

In yet other preferential conditions of implementation of the invention,
the bioreactor device and the automated selection device are supplied with the same substrate,
the bioreactor operating continuously, the substrate supply flow rate applied to the substrate inlet line is identical to that applied to the culture-medium extraction line.

An automated device for genetic selection of usable living cells C2 is in particular that described in WO-A-00/34433 and which can operate depending on culture conditions such that the selection device always favours the so-called “dynamic” variant living cells which are increasingly well-adapted to the culture conditions maintained in the bioreactor vessel.

In parallel with the operation of the bioreactor, all or part of the culture present in the automated selection device is periodically transferred to the bioreactor vessel.

In the invention, a reserve of dynamic variant living cells increasingly well-adapted to the pre-established culture conditions imposed in the bioreactor vessel is therefore permanently available.

The dynamic variant living cells with a strong growth rate selected by the selection device are inoculated periodically into the bioreactor vessel where they supplant the static living cells with a weaker growth rate present in the tank.

The ratio of the growth rate of the living cells present in the tank to that of the living cells with an increased growth rate selected by the selection device is such that the living cells originating from the selection device rapidly supplant the living cells present in the tank of the bioreactor.

In fact the growth rate of the living cells originating from the selection device is always at least equal to the maximum growth rate of the living cells present in the bioreactor vessel.

In summary, if, during continuous culture, the growth rate of the living cells originating from the automatic selection device is equal to the growth rate of the living cells present in the bioreactor vessel, then all of the living cells will develop at the same time; if it is greater, then the living cells originating from the automatic selection device will supplant those already present in the bioreactor vessel.

Moreover, by periodically transferring living cells from the bioreactor vessel to the automated selection device, there is the certainty of bringing the two populations of living cells into competition and selecting from the possible variants originating from the bioreactor vessel and those from the automated selection device the living cells best-adapted to the conditions in the bioreactor.

The performances of the bioconversion or biodegradation method carried out in the bioreactor vessel are therefore at worst maintained but usually constantly improved due to the periodic replacement of the active living cells present in the bioreactor vessel by active living cells originating from the automated selection device, which is always more efficient, being always better adapted to the culture conditions.

Moreover the inoculation being repeated periodically and consequently with living cells increasingly well-adapted to the culture conditions, the automated selection device guarantees the preponderance of the living cells which are most active vis-à-vis the substrate present in the bioreactor vessel.

A small automated selection device, for example equipped with culture vessels of only 25 ml, is sufficient to effectively operate a bioreactor vessel such as the 100 m³ aeration tank of a sewage treatment station. It is of course possible to use culture vessels with a larger volume, for example 1 litre.

The living cells C2 used which improve the bioconversion reaction can in particular be produced by implementation of a method comprising the following stages:
(a) making available a culture in at least one first culture vessel;
(b) continuous supply of the culture in the first culture vessel with gas from a gas source and regular replenishment with liquids from a source of medium,
(c) transfer of the culture from the first culture vessel by connecting pipes into at least one second culture vessel by means of an appropriate pipe circuit,
(d) connection of the first culture vessel to a source for a sterilizing agent, in order to sterilize the first culture vessel,
(e) removal of the sterilizing agent from the first culture vessel,
(f) continuous supply of the culture in the second culture vessel with gas from the source gas and regular replenishment with liquids from the source of medium,
(g) return of the culture from the second culture vessel via the connecting pipes into the first culture vessel by means of an appropriate pipe circuit,
(h) connection of the second culture vessel to the source for the sterilizing agent, in order to sterilize the second culture vessel; and
(i) removal of the sterilizing agent from the second culture vessel.

Under preferential conditions of implementation of the method described above, stages (b) to (h) are repeated at least once.

In the present Application and in what follows, the term “bioreactor vessel” denotes for example the aeration tank of a treatment plant, the methanization tank of an anaerobic biological treatment unit, a lagoon, a reservoir, a container for example from 0.5 litre to 100 m³, in particular from 1 litre to 100 m³, particularly from 5 litres to 50 m³ and quite particularly from 10 litres to 50 m³ or a fermenter for example from 0.5 litre 100 m³, in particular from 1 litre to 100 m³, particularly from 5 litres to 50 m³ and quite particularly from 10 litres to 50 m³.

In the present Application and in what follows, the term “substrate” denotes a medium containing a compound the metabolic conversion of which is envisaged, in particular a water of industrial origin such as for example water used for washing hydrocarbon storage tanks, water used for washing pharmaceutical-intermediate production installations, water used for rinsing filter cakes, water used for washing fumes originating from chemical production methods, effluents originating from the de-icing of aircraft, water of municipal origin such as for example domestic sewage, an accidental pollutant of the environment such as for example the presence in the sea of a slick of hydrocarbons or other chemicals originating respectively from the wreck of an oil or chemical tanker, chemical effluents spread on the ground following an accident involving a road or rail tanker, soils polluted with heavy metals or dioxin.

The term “substrate” also denotes a compound the metabolic conversion of which is envisaged, such as for example glucose used in the production of biomolecules of industrial interest such as lysin, xanthan, alginates, polyols such as glycerol, hygromycin, ethanol used in the production of vinegar by acetic fermentation, oxalic acid used for biohydrometallurgical applications, pectins and carrageenans.

The term “substrate” also denotes a medium containing living or dead cells the metabolic conversion of which is envisaged, for example an activated sludge of urban or industrial waste water, lignocellulosic derivatives originating from the paper industry, by-products in solid or paste form originating from the agri-food industry, for example vegetation biomass (in particular cut grass), malt husks, yeasts, molasses, or also by-products of the fishing industry such as chitinous derivatives, for example those originating from crab or shrimp shells.

The term “substrate” also denotes pollutant molecules such as volatile organochlorinated compounds (such as chlorinated solvents and the CFCs), organochlorinated pesticides (such as DDT); halogenated polycyclic aromatic hydrocarbons (such as the PCBs, dioxins and furans); solvents (such as benzene, toluene, xylene), organochlorinated phytosanitary compounds or organophosphates.

The term “living cells” denotes for example one or more bacterial strains such as Sphingomonas wittichii (which catalyzes the bioconversion of dioxin), Pseudomonas putida (which catalyzes the bioconversion of cyanides and cyanates), Agrobacterium radiobacter (which catalyzes the bioconversion of pesticides such as bromoxynil), certain Alcanivorax or Acinetobacter strains (capable of biodegrading numerous aliphatic hydrocarbons), Xanthomonas campestris (which is involved in the biosynthesis of xanthan) or Sphingomonas paucimobilis (which is involved in the biosynthesis of gellan).

The term “living cells” also denotes animal cells such as mammal cells (such as HEK-293 cells) for the production of monoclonal antibodies, cell growth factors of insect cells for the production of recombinant proteins or entomopathogenic viral particles (such as Sf9 cells).

The term “living cells” also denotes plant cells such as Datura plant cells for the production of tropane alkaloids (atropine, hyosciamine and scopolamine), transgenic plant cells for the production of molecules of industrial interest (such as the overproduction of starch by potatoes).

The term “living cells” also denotes algae such as the green algae belonging to the species Spirogyra involved in the biological treatment of effluents containing dyes such as Reactive Yellow 22, cultures of the micro-alga Scenedesmus quadricauda used in the bioconversion of progesterone, the micro-algae Chlorella vulgaris and Coenochloris pyrenoidosa involved in the biodegradation of p-chlorophenol, the macro-alga Microspora capable of eliminating lead.

The term “living cells” similarly denotes yeasts such as Saccharomyces cerevisiae used in the production of bioethanol from glucose or in the production of xylitol from glucose, Candida tropicalis YMEC14 used in the biodegradation of phenol compounds (originating from the production of olive oil), Candida famata used in the biodegradation of nitrilated composes.

The term “living cells” equally denotes fungi such as Penicillium janthinellum capable of producing a xylanase, an enzyme depolymerizing xylane, Streptomyces clavuligerus capable of producing cephalosporin C from glucose as the only source of carbon, or Phanerochaete chrysosporium capable of biodegrading the di-and tetrachlorinated dioxins.

The term “living cells” also denotes protozoa such as Euglena mutabilis (acidophilic protozoa) involved in the bioconversion of arsenic.

The term “living cells” also denotes a mixture of all the above-mentioned types of living cells.

Periodic inoculation originating from the automatic device for selecting living cells is for example carried out every 48 hours, preferably at least once a week, particularly at least once a month and quite particularly after each appreciable improvement in the growth rate of the living cells C2.

The methods for continuous, semi-continuous or discontinuous treatment of a substrate forming the subject of the present invention possess very useful qualities. They make it possible in particular to biologically control the operation of a bioreactor of standard design by controlling the living cells present by elimination of the living cells least adapted to the culture medium such as a contaminant having a growth rate lower than that of the living cells present in the bioreactor vessel for example. It is therefore possible to be freed from the constraints of sterility.

A small selection device, for example equipped with one-litre bioreactor vessels, is sufficient for the effective operation of a vessel such as a reservoir with a volume of 4000 m³.

The invention also makes it possible to improve the effectiveness of a culture method of standard design by increasing the activity of the living cells utilized in the method without reconstructing the devices used. It is thus possible to increase the production yields of a molecule of interest and/or the degradation rate of substrates.

These qualities are illustrated hereafter in the experimental part.

They justify the use of the methods described above for example in the biodegradation of compounds which are difficult to manage. In fact a great deal of waste originating from the chemicals industry is at present destroyed by incineration at high cost and with a significant environmental risk linked to the risk of emission into the atmosphere of compounds dangerous to humans and their environment. The biological treatment of this waste (or bioconversion) is often made impossible by the time required for treatment or by the inability of the living cells to metabolize the compounds presents in the waste, or also by the inhibiting effect of certain compounds vis-à-vis bacterial activity in general.

The device of the invention makes it possible to maintain, in a bioreactor vessel dedicated to the bioconversion of waste, living cells specifically adapted and effective vis-à-vis the compounds present in the waste and therefore to make possible the bioconversion of waste customarily destroyed by incineration.

By selecting living cells which are increasingly well-adapted to the culture medium by means of the selection device, the invention makes it possible to improve the effectiveness of a culture method of standard design by increasing the activity of the living cells utilized in the method without reconstructing the implementation devices.

These qualities also justify the use of the methods described above for example in improving the operation of biological effluent treatment stations. In fact, the satisfactory operation of urban or industrial effluent treatment plants can be affected by the accidental presence in the effluents of compounds which are difficult to manage vis-à-vis the living cells present. A remedy to this problem can be provided by the addition of a device such as described above.

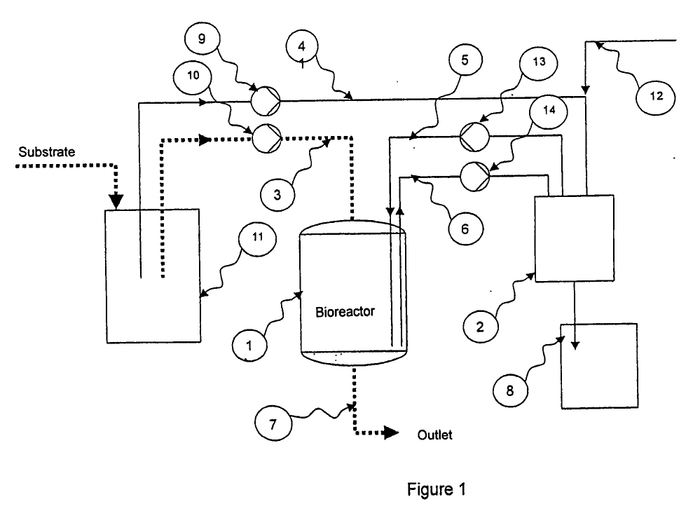

The bioreactor vessel is in this case formed by the existing aeration tank of the treatment plant. The automated selection device can be supplied via a connection piece situated upstream of the aeration system in a primary settling tank for example. The reciprocal inoculation of the selection device and the bioreactor vessel is carried out as illustrated in FIG. 1 below. It is also possible to use an external connection line for supplying the automatic device with a substrate modified with respect to the medium collected upstream of the aeration tank. This device can be used to enrich the aeration tanks with living cells adapted to the biodegradation of any compounds which are difficult to manage and are present in the effluents.

These qualities also justify the use of the methods described above, for example in improving the performances of the biosyntheses. To this end the invention can be used to improve the performances (yield, growth time) of industrial synthesis methods by biocatalysis.

At present, the improvement in the performances of the biosyntheses based on fermentation methods, whether batch or continuous, is achieved by optimizing the composition of the culture medium and physico-chemical parameters of the culture (temperature, oxygenation, pH, etc.).

These developments take a long time, are expensive and in any case limited by the metabolism of the living cells present.

The invention, by acting on the metabolism of the living cells present makes it possible to adapt said living cells to the conditions imposed by the technical and economic imperatives and to increase the growth rate, and thus consequently to increase the overall biosynthesis performances.

In the case for example of so-called osmotolerant yeasts involved in the production of the polyols (sorbitol, mannitol, xylitol etc.), culture times varying from 4 to 5 days are observed in glucose concentrations of approximately 30 g/l.

By means of the invention it is possible to bring the yeasts into contact with increasing concentrations of glucose so as to orient their natural metabolism towards the production of the desired extracellular or intracellular metabolite, while increasing their growth rate; i.e. reducing the time necessary for the culture.

This improvement is reflected in an increase in the productivity of the equipment used and by a reduction in the cost of the biosynthesis.

The method according to the invention can be implemented over long periods and even indefinitely.

A subject of the present Application is also a device for culturing living cells comprising:
A: a selection device preferably comprising
   two vessels or more making it possible to receive and maintain cultures of living cells in suspension,
   a set of means making it possible to separately supply these vessels with sterilization, cleaning or neutralization fluids,
   a set of means making it possible to supply these vessels with gas,
   a set of means making it possible to supply these vessels with substrate,
   a set of means making it possible to transfer the content of one vessel into the other and vice-versa,
   optionally a set of means making it possible to evacuate all or part of the content of these vessels to another device such as a bioreactor vessel,
   a set of means making it possible to evacuate all or part of the content of these vessels to a refuse bin.
B: a bioreactor vessel,
C: a system of means for transferring living cells between the selection device and the bioreactor vessel,
D: optionally a pipe comprising means for connecting the bioreactor vessel to a solid-liquid separation device such as a settling tank,
E: optionally a pipe for evacuation of the fluid (water for example) treated,
F: optionally a temperature regulation device.

The means for transferring the content of one vessel to the other and vice-versa can be physical means such as pipes or human means making collections from one to be transferred into the other.

A more particular subject of the present Application is a device for culturing living cells by coupling with an automatic device for selecting living cells comprising:
A: a device for selecting living cells comprising
   (a) at least one first and at least one second culture vessel intended to receive a culture,
   (b) a source of gas,
   (c) a source of medium,
   (d) a source for a sterilizing agent; and
   (e) a system of pipes comprising means for connecting either one of the two culture vessels to the source of medium such as valves as well as connecting the two culture vessels to each other and for connecting either one of the other culture vessels to the source of the sterilizing agent.
B: a bioreactor vessel
C: a system of means for transferring living cells between the selection device and the bioreactor vessel,
D: optionally a pipe comprising means for connecting the bioreactor vessel to a solid-liquid separation device such as a settling tank
E: optionally a pipe for evacuation of the fluid (water for example) treated.
F: optionally a temperature regulation device.

Under preferential conditions for implementation of the invention,
an extraction line is installed between the bioreactor vessel and the automated selection device to make it possible to collect living cells present in the bioreactor vessel in order to make them develop in the automated selection device,
an inoculation line is installed between the automated selection device and the bioreactor vessel to make it possible to seed the bioreactor vessel in repeated and regular manner with living cells which have developed in the automated selection device,
an additional line makes it possible to enrich the culture medium of the automatic device with one or more additives,
a reservoir for collecting the rinsing and sterilization effluents makes it possible to collect the sterilizing and rinsing liquids from the automated selection device,
a set of pumps allows the transfer of the different fluids.

The preferential conditions for implementation of the methods described above also apply to the other subjects of the invention referred to above, in particular to the devices for their implementation.

The invention will be better understood by referring to the attached drawings in which

FIG. 1 represents a diagrammatic view of a device of the invention,

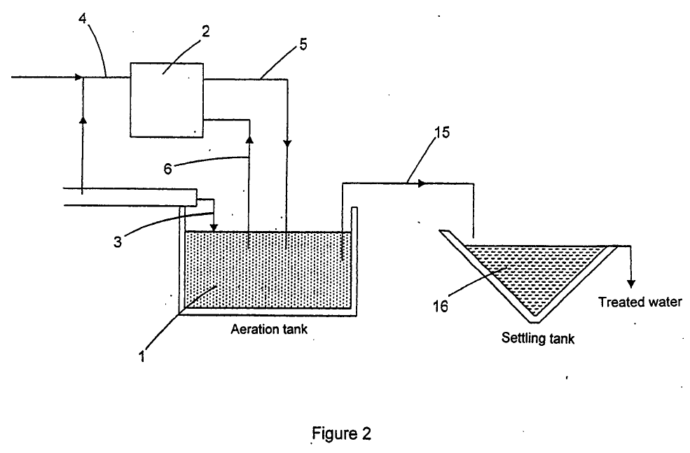

FIG. 2 represents a diagrammatic view of a biological sewage-purification device,

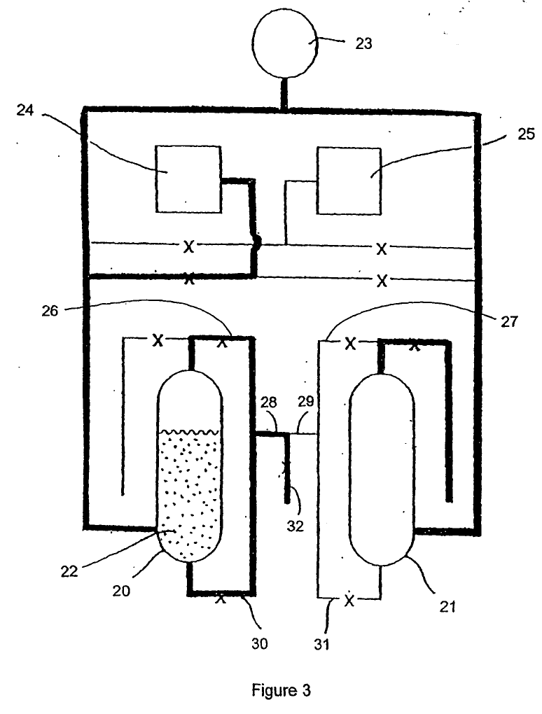

FIG. 3 represents a diagrammatic view of an automated selection device described in WO 00/34433.

FIG. 1 shows a bioreactor vessel 1 connected by a system of return 5 and outward 6 pipes to an automated culture selection device 2. Pumps 13,14 are provided on these pipes.

A surge tank 11 of substrate can also be seen which is supplied externally with substrate and connected by a system of pipes 4 on the one hand to the automated culture selection device 2 and on the other hand to the bioreactor vessel 1. Pumps 9, 10 are provided on these pipes.

A tank 8 for collecting rinsing and sterilization effluents from the automated device 2 for selecting living cells is provided.

An inlet pipe 12 for additives is provided for addition to the automated device 2 for selecting living cells.

The device can in particular operate as follows:

The bioreactor vessel 1 and the automated selection device 2 are supplied with the same substrate by routes 3 and 4 respectively.

The bioreactor 1 operating continuously, the substrate supply flow rate applied to the line 3 is identical to that applied to the line 7 corresponding to the extraction of culture medium. The line 7 can lead to a solid-liquid separation device, not shown, such as a settling tank.

An inoculation line 5 installed between the automated selection device 2 and the bioreactor vessel 1 makes it possible to seed the bioreactor vessel 1 in repeated and regular manner with living cells having developed in the automated selection device 2.

An extraction line 6 installed between the bioreactor vessel 1 and the automated selection device 2 makes it possible to collect living cells present in the bioreactor vessel 1 in order to develop them in the automated selection device 2.

An additional line 12 makes it possible to enrich the culture medium of the automatic device with one or more additives.

A refuse bin 8 makes it possible to collect the sterilizing and rinsing from the automated selection device.

An set of pumps 9, 10, 13 and 14 allows transfer of the different liquids.

FIG. 2 shows a biological sewage-purification device.

It shows some of the elements above, namely a bioreactor vessel 1 which is an aeration tank and an automated selection device 2 supplied with the same substrate by routes 3 and 4 respectively, inoculation and extraction lines 5 and 6 installed between the bioreactor vessel 1 and the automated selection device 2, and a pipe 15 comprising means for connecting the bioreactor vessel 1 to a solid-liquid separation device, in the present case a settling tank 16.

FIG. 3 shows a first and a second culture vessel 20, 21, intended to receive a culture 22, a source of gas 23, a source of medium 24, a source 25 for a sterilizing agent, and a system of pipes comprising means for connecting either one of the two culture vessels 20 or 21 to the source of medium 24 such as valves as well as connecting the two culture vessels 20, 21 to each other and for connecting either of the other culture vessels 20 or 21 to the source 25 of the sterilizing agent. The bold lines represent the pipes active during one of the phases of implementation of the method.

This device allows the provision of a culture 22 to at least one first culture vessel 20, the continuous supplying of the culture 22 in the first culture vessel 20 with gas from a source of gas 23 and regular replenishment with liquids from a source of medium 24, the transfer of the culture 22 from the first culture vessel 20 by connecting pipes 28-31 into at least one second culture vessel 21 by means of an appropriate pipe circuit, the connection of the first culture vessel 20 to a source 25 for a sterilizing agent, to sterilize the first culture vessel 20, the removal of the sterilizing agent from the first culture vessel 20, the continuous supplying of the culture 22 in the second culture vessel 21 with gas from the source of gas 23 and regular replenishment with liquids from the source of medium 24, the return of the culture 22 from the second culture vessel 21 via the connecting pipes 28-31 into the first culture vessel 20 by means of an appropriate pipe circuit, the connection of the second culture vessel 21 to the source 25 for the sterilizing agent for sterilizing the second culture vessel 21 and for the removal of the sterilizing agent from the second culture vessel 21.

The examples which follow illustrate the present Application.

### EXAMPLE 1

### Bioconversion of Waste Originating from the Production of Pesticides

A bioreactor vessel with a useful capacity of 5 litres is continuously supplied, at a fixed flow rate of 0.75 ml/min, with a substrate comprising waste originating from the production of pesticides. Analysis of this waste reveals the following chemical compounds: alcohols (e.g. 2-butoxyethanol), alkanes (e.g. propane-2,2-dimethoxy), chlorophenols (e.g. 2,4-dichlorophenol), aromatics (e.g. 1,1′-biphenyl, 1-methylnaphthalene, 2-methylnaphthalene, 2-ethylnaphthalene), brominated compounds (e.g. benzonitrile-3,3-dibromo-4-hydroxy) and pesticides (e.g. 2,4-D-butoxyethylester, MCP and MCPP). The chemical oxygen demand of this waste is 2450 mg/l. The bioreactor vessel is inoculated with 10 ml of a mixture of living cells of microorganisms isolated from samples originating from different ecological niches or activated sludges from treatment plants; these living cells are selected because they are capable of degrading the waste.

The continuous regime is maintained by fixing a residual COD of 1000 mg/l, which represents a bioconversion yield stabilized at 59.18%.

Moreover, an automated selection device of the type described in FIG. 1 of WO-A-00/34433 equipped with 25 ml culture vessels is also supplied with the same substrate and inoculated with the same mixture of living cells as previously. At the start, the same living cells are therefore present in both vessels.

Once the turbidity setting is reached for the selection system (detection with the turbidostat), the inoculation of the bioreactor vessel with 10 ml of the medium present in the automated selection device follows automatically.

After operation for 9 weeks, it is noted that the living cells originating from the automated selection device, the growth rate of which has doubled during this period (passing from 0.009 to 0.018 h⁻¹), have replaced the population of living cells originally present in the bioreactor vessel.

At this final stage, it has only been possible to define two microorganisms as being Delftia acidovorans and Pseudomonas putida A.

It has therefore been possible to increase the supply flow rate to the fermenter while maintaining the same bioconversion yield.

1-12. (canceled)

13. Method for the continuous, semi-continuous or discontinuous treatment of a substrate (24), in which said substrate (24) installed in a bioreactor vessel (1) is subjected to the action of a culture of living cells C1 making it possible to carry out a reaction R1 on said substrate (24), characterized in that it comprises the following steps:
a) removing a population of living cells C1 present in the bioreactor vessel (1) in order to transfer them into an automatic device (2) for selecting living cells proliferating in suspension;
b) selecting the cells C2 proliferating in suspension in said device (2) by eliminating the static living cells, and
c) transferring all or part of cells C2 present in said selection device (2) periodically to the bioreactor vessel (1).

14. Method according to claim 13, characterized in that the automatic device (2) for selecting cells proliferating in suspension comprises:
two or more vessels (20, 21) making it possible to receive and maintain cultures of living cells in suspension,
a set of means making it possible to separately supply these vessels with sterilizing (25), cleaning or neutralizing fluids,
a set of means making it possible to supply these vessels with gas (23),
a set of means making it possible to supply these vessels with substrate (24),
a set of means (28-31) making it possible to transfer the content of one vessel (20) into the other (21) and vice-versa,
a set of means making it possible to evacuate all or part of the content of these vessels to another device such as a bioreactor vessel (1),
a set of means making it possible to evacuate all or part of the content of these vessels (20, 21) to a refuse bin.

15. Method according to claim 13, characterized in that the automatic device for selecting cells proliferating in suspension comprises in particular
(a) at least one first and at least one second culture vessel (20, 21) intended to receive a culture (22),
(b) a source of gas (23);
(c) a source of medium (substrate) (24);
(d) a source (25) for a sterilizing agent; and
(e) a system of pipes comprising means for connecting either one of the two culture vessels (20 or 21) to the source of medium (24) such as valves as well as for connecting the two culture vessels (20, 21) to each other and for connecting either of the other culture vessels (20 or 21) to the source (25) of the sterilizing agent.

16. Method according to claim 13, characterized in that the living cells C2 originate from the selection carried out from a population of living cells proliferating exclusively in suspension.

17. Method according to claim 13, characterized in that the bioreactor vessel (1) is chosen from an aeration tank of a treatment plant, a methanization tank, an anaerobic biological treatment unit, a lagoon, a reservoir, a tank for example from 0.5 litre to 100 m³ or a fermenter.

18. Method according to claim 13, characterized in that the living cells C2 proliferating in suspension can in particular be produced by implementation of a method comprising the following stages:
(a) making available a culture (22) in at least one first culture vessel (20);
(b) continuous supplying of the culture (22) in the first culture vessel (20) with gas from a source of gas (23) and regular replenishment with liquids from a source of substrate (24),
(c) transfer of the culture (22) from the first culture vessel (20) by connecting pipes (28-31) into at least one second culture vessel (21) by means of an appropriate pipe circuit,
(d) connection of the first culture vessel (20) to a source (25) for a sterilizing agent, in order to sterilize the first culture vessel (20),
(e) removal of the sterilizing agent from the first culture vessel (20),
(f) continuous supplying of the culture (22) in the second culture vessel (21) with gas from the source of gas (23) and regular replenishment with liquids from the source of medium (24),
(g) return of the culture (22) from the second culture vessel (21) via the connecting pipes (28-31) into the first culture vessel (20) by means of an appropriate pipe circuit,
(h) connection of the second culture vessel (21) to the source (25) for the sterilizing agent, in order to sterilize the second culture vessel (21); and
(i) removal of the sterilizing agent from the second culture vessel (21).

19. Method according to claim 13, characterized in that the substrate (24) is chosen from a medium containing a compound the metabolic conversion of which is envisaged, water of industrial origin, domestic sewage, an accidental pollutant of the environment such as the presence in the sea of a slick of hydrocarbons or other chemical products, chemical effluents spread on the ground, soil polluted with heavy metals or dioxin, a compound the metabolic conversion of which is envisaged such as glucose, ethanol or oxalic acid, a volatile organochlorinated compound, an organochlorinated pesticide, a halogenated polycyclic aromatic hydrocarbon or a solvent.

20. Method according to claim 13, characterized in that the living cells comprise one or more species chosen from bacteria, animal or plant cells, cells of algae, yeasts or fungi.

21. Method according to claim 13, characterized in that the transfer originating from the automatic device (2) for selecting living cells envisaged in stage c) is carried out at least once a week.

22. Device for culturing living cells characterized in that it comprises:
A: a bioreactor vessel (1) capable of containing a population of living cells C1 carrying out a reaction R1 of bioconversion of a substrate (24);
B: an automatic device (2) for selecting living cells proliferating in suspension, making it possible to select living cells C2 proliferating in suspension by elimination of the static living cells, this device comprising a set of means making it possible to supply of said device with sterilization, washing or neutralizing liquids;
C: a system of pipes (5) comprising means for carrying out transfers from the selection device (2) to the bioreactor vessel (1); and
D: a system of pipes (6) comprising means for carrying out transfers from the bioreactor vessel (1) to the selection device (2).

23. Device for culturing living cells according to claim 22, characterized in that it comprises, in addition:
E: a pipe (15) comprising means for connecting the bioreactor vessel (1) to a solid-liquid separation device such as a settling tank (16).

24. Device for culturing living cells according to claim 22, characterized in that the selection device (2) comprises:
two or more vessels (20, 21) making it possible to receive and maintain cultures of living cells in suspension,
a set of means making it possible to supply these vessels with substrate (24),
a set of means (28-31) making it possible to transfer the content of one vessel (20) into the other (21) and vice-versa,
a set of means making it possible to evacuate all or part of the content of these vessels to another device such as a bioreactor vessel (1),
a set of means making it possible to evacuate all or part of the content of these vessels (20, 21) to a refuse bin.

25. Device for culturing living cells according to claim 22, characterized in that the device for selecting living cells proliferating in suspension comprises:
(a) at least one first and at least one second culture vessel (20, 21) intended to receive a culture (22),
(b) a source of gas (23),
(c) a source of medium (2),
(d) a source (25) for a sterilizing agent; and
(e) a system of pipes comprising means for connecting either one of the two culture vessels (20 or 21) to the source of medium (24) such as valves as well as connecting the two culture vessels (20, 21) to each other and for connecting either one of the other culture vessels (20 or 21) to the source (25) of the sterilizing agent.

26. Device according to claim 22, characterized in that the bioreactor vessel (1) is chosen from an aeration tank of a treatment plant, a methanization tank of an anaerobic biological treatment unit, a lagoon, a reservoir or a tank from 0.5 litre to 100 m³.
